(19) United States
(12) Patent Application Publication
(10) Pub. No.: US 2021/0163541 A1
(43) Pub. Date: Jun. 3, 2021

(54) Method for Preparing PNA Oligomer

(71) Applicant: SEASUNBIO MATERIALS, Daejeon (KR)

(72) Inventors: Heekyung Park, Daejeon (KR); In Seok Hong, Sejong-si (KR); Goon Ho Joe, Daejeon (KR); Yongtae Kim, Sejong-si (KR)

(21) Appl. No.: 17/255,478

(22) PCT Filed: Jun. 27, 2019
(86) PCT No.: PCT/KR2019/007823

§ 371 Date: Dec. 23, 2020

(30) Foreign Application Priority Data
Jun. 27, 2018 (KR) 10-2018-0074111
Jun. 26, 2019 (KR) 10-2019-0076555

(51) Int. Cl. C07K 14/00

(52) U.S. Cl. CPC C07K 14/003

(57) ABSTRACT

The present invention provides a method for preparing a PNA oligomer. More specifically, the present invention can prepare a PNA oligomer which is easily separable from byproducts through a simple and short process by using PNA dimers, PNA trimers or PNA tetramers, and which has extremely high yields and purity.

### CROSS-REFERENCE TO RELATED APPLICATIONS

This application is the United States national phase of International Application No. PCT/KR2019/007823 filed Jun. 27, 2019, and claims priority to Korean Patent Application Nos. 10-2018-0074111 filed Jun. 27, 2018 and 10-2019-0076555 filed Jun. 26, 2019, the disclosures of which are hereby incorporated by reference in their entirety.

### BACKGROUND OF THE INVENTION

### Field of the Invention

The present invention relates to a method for preparing a PNA oligomer.

### Description of Related Art

As is known, nucleic acids are DNA and RNA which are responsible for the genetic information of an organism.

On the other hand, peptide nucleic acid (PNA) is a nucleic acid obtained by converting a sugar-phosphate skeleton of a nucleic acid to an N-(2-aminoethyl)glycine skeleton.

Sugar-phosphate skeletons of DNA/RNA have negative charges under neutral conditions, resulting in electrostatic repulsion between complementary chains. However, since a backbone structure of PNA does not inherently have a charge, there is no electrostatic repulsion.

That is, a main chain of PNA has virtually no charge at all, which is a very important feature of PNA. Due to such a feature, PNA may be used for various purposes for which natural oligonucleotides or oligonucleotide derivatives are difficult to use.

Furthermore, PNA binds to DNA or RNA with a higher affinity than natural oligonucleotides and is very stable in serum in comparison to natural DNA.

However, although interests and studies on the use of PNA have been diversified and increased, studies on a method for synthesizing a PNA oligomer have not been actively conducted.

A solid-phase peptide synthesis method is generally used in the method for synthesizing a PNA oligomer. Therefore, when a PNA monomer unit is classified by a skeleton structure of PNA, the PNA monomer unit may be classified into two types of an Fmoc-type PNA monomer unit and a Boc-type PNA monomer unit.

A method for synthesizing the Fmoc-type PNA monomer unit has already been established, but there are problems in that mass production is not easy and a yield and a purity are low.

As an example, WO 2005-009998 A1 discloses a monomer capable of easily synthesizing PNA with a high yield, but it is not cost-effective in mass production due to many processes.

Therefore, a method for preparing a PNA oligomer capable of efficiently preparing a desired PNA with a high purity and yield through a simple process is required.

### SUMMARY OF THE INVENTION

An object of the present invention is to provide a method for preparing a desired PNA oligomer with a remarkably increased purity and yield while implementing significant cost-effectiveness through remarkably reduced process steps.

### Technical Solution

The present invention provides a method for preparing a PNA oligomer with an extremely increased purity and yield through a simple process. In one general aspect, a method for preparing a PNA oligomer includes:

a step a) of preparing a structure represented by the following Formula 2, 3, or 4 by binding a first PNA dimer, a first PNA trimer, or a first PNA tetramer to a structure represented by the following Formula 1; and

a step b) of preparing a PNA oligomer represented by the following Formula 4, 5, or 6 by binding a second PNA dimer, a second trimer PNA, and a second tetramer PNA to the structure represented by Formula 2, 3, or 4,

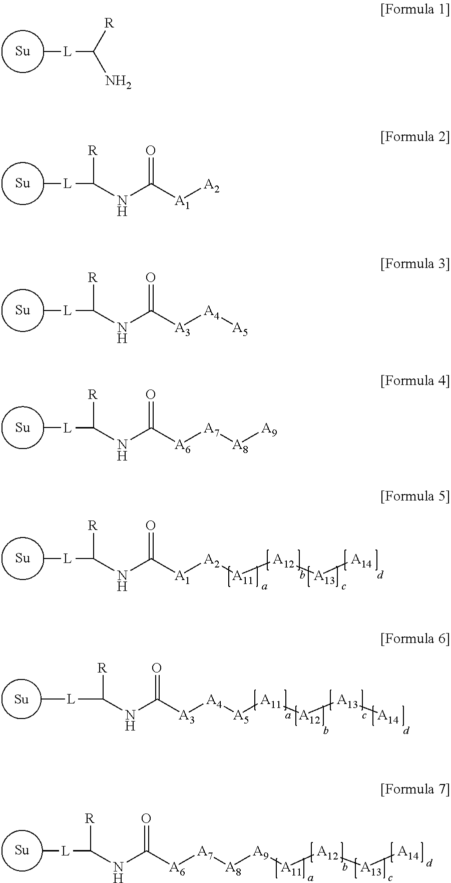

in Formulas 1 to 7,

Su represents a support,

L and R each independently represent hydrocarbyl or heterohydrocarbyl,

A₁ to A₉ and A₁₁ to A₁₄ each independently represent a PNA monomer containing identical or different nucleic acid bases, and

a and b each independently represent an integer of 1, and c and d independently represent an integer of 0 or 1.

More preferably, according to an embodiment of the present invention, the method for preparing a PNA oligomer may include: a step A) of obtaining a product that binds a first PNA dimer in which an amine group is protected, a first PNA trimer in which an amine group is protected, or a first PNA tetramer in which an amine group is protected to the structure represented by Formula 1;

a step B) of preparing a deprotected amine product by deprotecting the amine group of the product obtained in the step A); and

a step C) of binding a second PNA dimer in which an amine group is protected, a second PNA trimer in which an amine group is protected, or a second PNA tetramer in which an amine group is protected to the deprotected amine product.

Preferably, according to an embodiment of the present invention, the method for preparing a PNA oligomer may further include a step of repeatedly performing the step B) and the step C), and according to an embodiment of the present invention, the PNA oligomer may contain four or more nucleic acid bases.

According to an embodiment of the present invention, the first PNA dimer, the first PNA trimer, or the first PNA tetramer may be used in an amount of 2 to 5 equivalents with respect to 1 equivalent of an amine functional group of Formula 1.

According to an embodiment of the present invention, the binding in the step b) may be performed by a coupling reaction using N,N,N′,N′-tetramethyl-O-(1H-benzotriazol-1-yl)uronium hexafluorophosphate (HBTU) and benzotriazol-1-yloxy)tripyrrolidinophosphonium hexafluorophosphate (PyBop), and

the HBTU and the PyBop may be used in an equivalent ratio of 1:1 to 3.

Preferably, according to an embodiment of the present invention, the binding in the step b) may be performed in a mixed solvent of chlorinated (C1-C4) alkane, dimethylformamide (DMF), and N,N-diisopropylethylamine (DIEA), and the chlorinated (C1-C4) alkane may be one or two or more selected from trichloromethane, dichloromethane, chloromethane, 1,1,2-trichloroethane, 1,1,1-trichloroethane, 1,2-dichloroethane, 1,1-dichloroethane, and chloroethane.

According to an embodiment of the present invention, the DIEA may be contained in the mixed solvent in an amount of 10 wt % with respect to a total weight of the mixed solvent, and a volume ratio of the chlorinated (C1-C4) alkane to the DMF may be 1:1 to 2.

According to an embodiment of the present invention, the first PNA dimer may contain a nucleic acid base identical to or different from that of the second PNA dimer, the first PNA trimer may contain a nucleic acid base identical to or different from that of the second PNA trimer, and the first PNA tetramer may contain a nucleic acid base identical to or different from that of the second PNA tetramer.

According to an embodiment of the present invention, the nucleic acid base may be adenine, cytosine, 5-methylcytosine, guanine, thymine, uracil, purine, 2,6-diaminopurine, N⁴N⁴-ethanocytosine, N⁶N⁶-ethano-2,6-diaminopurine, 5-(C3-C6)-alkynyluracil, 5-(C3-C6)-alkynyl-cytosine, 5-(1-propargylamino)uracil, 5-(1-propargylamino)cytosine, phenoxazine, 9-aminoethoxyphenoxazine, 5-fluorouracil, pseudoisocytosine, 5-(hydroxymethyl)uracil, 5-aminouracil, pseudouracil, dihydrouracil, 5-(C1-C6)-alkyluracil, 5-(C1-C6)-alkyl-cytosine, 5-(C2-C6)-alkenylcytosine, 5-fluorocytosine, 5-chlorouracil, 5-chlorocytosine, 5-bromouracil, 5-bromocytosine, 7-deazaadenine, 7-deazaguanine, 8-azapurine, 7-deaza-7-substituted purine, thiouracil, or an artificial nucleic acid base, but is not limited thereto.

Preferably, according to an embodiment of the present invention, the nucleic acid base may have one or more amine protective groups, and a preferred amine protective group may be fluorenylmethoxycarbonyl (Fmoc), tert-butyloxycarbonyl (Boc), benzyloxycarbonyl (Cbz), benzhydryloxycarbonyl (Bhoc), acetyl, benzoyl, benzyl, carbamate, p-methoxybenzyl, 3,4-dimethoxybenzyl, p-methoxyphenyl, tosyl, trichloroethyl chloroformate, sulfonamides (Nosyl & Nps), or isobutyryl.

Preferably, according to an embodiment of the present invention, the first PNA dimer or the second PNA dimer may be represented by the following Formula 11, the first PNA trimer or the second PNA trimer may be represented by the following Formula 12, and the first PNA tetramer or the second PNA tetramer may be represented by the following Formula 13,

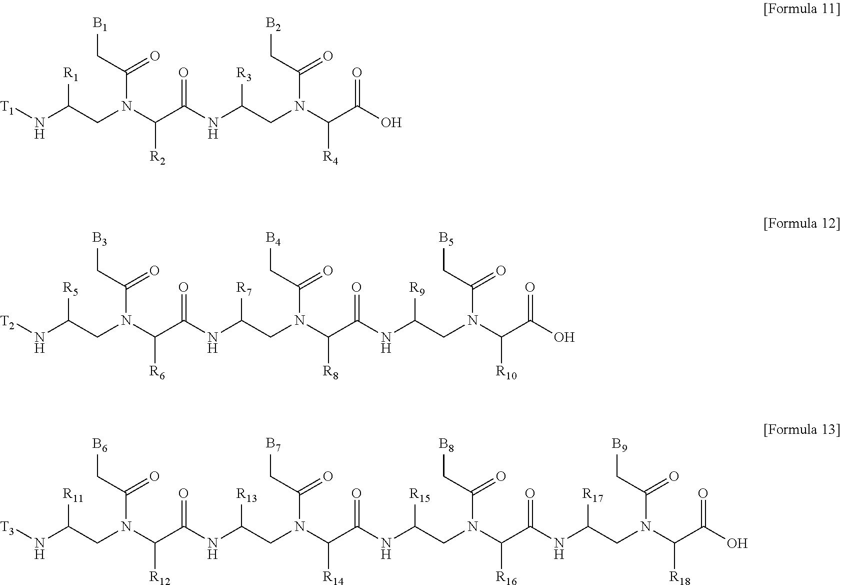

in Formulas 11 to 13,

R₁ to R₁₈ each independently represent hydrogen, an amino acid residue, or an amino acid residue having a substituent,

T₁ to T₃ each independently represent an amine protective group, and

B₁ to B₉ each independently represent a nucleic acid base having or not having an amine protective group.

Preferably, according to an embodiment of the present invention, the first PNA dimer, the second PNA dimer, the first PNA trimer, the second PNA trimer, the first PNA tetramer, and the second PNA tetramer may be prepared under a solution process or from a solid phase, and the support may be methylbenzhydrylamine (MBHA), a resin obtained by chloromethylating polystyrene (merrifield resin), a merrifield resin modified with 4-hydroxybenzyl alcohol (wang resin), a Boc-amino acid-linker bonded aminomethyl resin (PAM resin), an N-Fmoc-N-methoxy-linker bonded aminomethyl resin (weinreb resin), a resin obtained by binding p-nitrobenzophenone oxime to polystyrene (oxime resin), or a resin tritylated using polystyrene (trityl resin), but is not limited thereto.

According to an embodiment of the present invention, in the preparation method, when the number of nucleic acid bases of the prepared PNA oligomer is n, impurities of PNA oligomers containing n-1 and n-2 nucleic acid bases may not be present.

### Advantageous Effects

In the method for preparing a PNA oligomer of the present invention, the PNA dimer, the PNA trimer, or the PNA tetramer is used, such that the PNA oligomer may be prepared through a simpler process and a desired PNA oligomer may be more accurately prepared, compared to the method using a PNA monomer according to the related art.

Further, in the method for preparing a PNA oligomer of the present invention, the PNA oligomer is prepared through a shorter process step than in the method using the PNA monomer according to the related art, and the yield and purity of the prepared PNA oligomer are extremely high due to its very easy separation from by-products.

Further, in the method for preparing a PNA oligomer of the present invention, a very small amount of the PNA dimer, the PNA trimer, or the PNA tetramer is used as compared to the method according to the related art, such that significant cost-effectiveness is implemented.

### BRIEF DESCRIPTION OF DRAWINGS

FIG. 1 is a view illustrating an HPLC result of a mixed solution of a 12mer crude PNA oligomer and a 15mer crude PNA oligomer prepared in Example 16 of the present invention.

FIG. 2 is a view illustrating a comparison between a method for synthesizing a PNA oligomer using a PNA trimer of the present invention and a method for synthesizing a PNA oligomer according to the related art.

### DESCRIPTION OF THE INVENTION

Hereinafter, a method for preparing a PNA oligomer of the present invention and the PNA oligomer prepared using the same will be described. However, technical terms and scientific terms used herein have the general meanings understood by those skilled in the art to which the present invention pertains unless otherwise defined, and a description for the known function and configuration unnecessarily obscuring the gist of the present invention will be omitted in the following description.

The term “amino acid” described herein is used in the broadest sense. Examples thereof include not only natural amino acids such as serine (Ser), asparagine (Asn), valine (Val), leucine (Leu), isoleucine (Ile), alanine (Ala), tyrosine (Tyr), glycine (Gly), lysine (Lys), arginine (Arg), histidine (His), aspartic acid (Asp), glutamic acid (Glu), glutamine (Gln), threonine (Thr), cysteine (Cys), methionine (Met), phenylalanine (Phe), tryptophan (Trp), and proline (Pro), but also unnatural amino acids such as amino acid mutants and derivatives. Taking into consideration such broad definitions, those skilled in the art could understand that examples of the amino acid herein may include L-amino acids; D-amino acids; chemically modified amino acids such as amino acid mutants and derivatives; amino acids that do not become constituent materials for proteins in a living body, such as norleucine, β-alanine, and ornithine; and chemically synthesized compounds having amino acid properties known to those skilled in the art. Examples of the unnatural amino acid may include α-methylamino acids (α-methylalanine and the like), D-amino acids, histidine-like amino acids (2-amino-histidine, β-hydroxy-histidine, homohistidine, α-fluoromethyl-histidine, α-methyl-histidine, and the like), amino acids having an extra methylene at a side chain thereof (“homo” amino acids), and amino acids in which a carboxylic acid functional group at a side chain is substituted with a sulfonic acid group (cysteic acid and the like), in addition to a threonine derivative A.

The term “amino acid residue having a substituent” described herein means that an amino acid residue has a substituent. Examples thereof may include alanine, cysteine, aspartic acid, glutamic acid, phenylalanine, glycine, histidine, isoleucine, lysine, leucine, methionine, asparagine, proline, glutamine, arginine, serine, valine, tryptophan, or tyrosine substituted with an acetyl group, and a peptide to which an amino acid is bound.

The term “protective group” described in the present invention refers to a functional group for protecting a specific functional group in an organic reaction, and any functional group may be used as long as it is a functional group within a range that can be recognized by those skilled in the art of organic synthesis. Specific examples of an amine protective group may include fluorenylmethoxycarbonyl (Fmoc), tert-butyloxycarbonyl (Boc), benzyloxycarbonyl (Cbz), Bhoc, allyloxycarbonyl (Alloc), acetyl, benzoyl, benzyl, carbamate, p-methoxybenzyl, 3,4-dimethoxybenzyl, p-methoxyphenyl, tosyl, trichloroethyl chloroformate, sulfonamides (Bts and Nosyl & Nps), and isobutyryl.

The term “hydrocarbyl” or “heterohydrocarbyl” described in the present invention refers to a radical having one bonding site derived from hydrocarbon or heterohydrocarbon, and “hetero” means that carbon is substituted with one or more hetero atoms selected from O, S, and N atoms.

The term “chlorinated alkane” described in the present invention means that one or more hydrogens of alkane are substituted with chloro. The alkane includes both a linear shape and a branched shape. Except as specifically stated, the alkane has 1 to 10 carbon atoms, preferably 1 to 7 carbon atoms, and more preferably 1 to 4 carbon atoms.

Unless otherwise specified with respect to “substituted”, “has a substituent”, and a substituent described herein, as an optionally substituted substituent of the present invention, halogen, hydroxyl, a carboxylic acid group, nitro, cyano, (lower) alkyl, haloalkyl, mono- or di-alkylamino, alkoxy, thioalkyl, cycloalkyl, heterocycloalkyl, aryl, heteroaryl, —NO₂, —NRₐ₁R_{b1}, —NRₐ₁C(═O)R_{b1}, —NRₐ₁C(═O)NRₐ₁R_{b1}, —NRₐ₁C(═O)OR_{b1}, —NRₐ₁SO₂R_{b1}, —ORₐ₁, —CN, —C(═O)Rₐ₁, —C(═O)ORₐ₁, —C(═O)NRₐ₁R_{b1}, —OC(═O)Rₐ₁, —OC(═O)ORₐ₁, —OC(═O)NRₐ₁R_{b1}, —NRₐ₁SO₂R_{b1}, —PO₃Rₐ₁, —PO(ORₐ₁)(OR_{b1}), —SO₂Rₐ₁, —S(O)Rₐ₁, —SO(NRₐ₁)R_{b1} (for example, sulfoximine), —S(NRₐ₁)R_{b1} (for example, sulfilimine), and —SRₐ₁ may be used, wherein Rₐ₁ and R_{b1} may be the same as or different from each other or may be each independently hydrogen, halogen, amino, alkyl, alkoxyalkyl, haloalkyl, aryl, or heterocycle, or Rₐ₁ and R_{b1} may be in a form of heterocycle together with an attached nitrogen atom. Here, Rₐ₁ and R_{b1} may be plural depending on atoms bound thereto. Preferably, the alkyl may be C₁₋₆ alkyl, the cycloalkyl and heterocycloalkyl may be C₃₋₁₂ cycloalkyl and C₃₋₁₂ heterocycloalkyl, respectively, the aryl may be C₆₋₁₂ aryl, and the heterocycle and heteroaryl may be C₃₋₁₂ heterocycle and C₃₋₁₂ heteroaryl, respectively.

The PNA monomer described in the present invention may contain a nucleic acid base having or not having an amine protective group at a basic skeleton of PNA, specifically, at the N-position of N-(2-aminoethyl)glycine (Compound 5) or Compound 8.

The PNA dimer described in the present invention is obtained by linking two PNA monomers in which a nucleic acid base is linked to a basic skeleton of PNA, which is the N-position of N-(2-aminoethyl)glycine (Compound 5) or Compound 8. The nucleic acid bases contained in the PNA dimer may be the same as or different from each other, and specifically, may be represented by Formula 11.

The PNA trimer described in the present invention is obtained by linking three PNA monomers in which a nucleic acid base is linked to a basic skeleton of PNA, which is the N-position of N-(2-aminoethyl)glycine (Compound 5) or Compound 8. The nucleic acid bases contained in the PNA trimer may be the same as or different from each other, and specifically, may be represented by Formula 12.

The PNA tetramer described in the present invention has a structure in which four PNA monomers are linked similarly to the PNA trimer, and specifically, may be represented by Formula 13.

The present invention provides a method for preparing a PNA oligomer with a high purity and yield. The method for preparing a PNA oligomer of the present invention includes:

a step a) of preparing a structure represented by the following Formula 2, 3, or 4 by binding a first PNA dimer, a first PNA trimer, or a first PNA tetramer to a structure represented by the following Formula 1; and

a step b) of preparing a PNA oligomer represented by the following Formula 4, 5, or 6 by binding a second PNA dimer, a second trimer PNA, and a second tetramer PNA to the structure represented by Formula 2, 3, or 4,

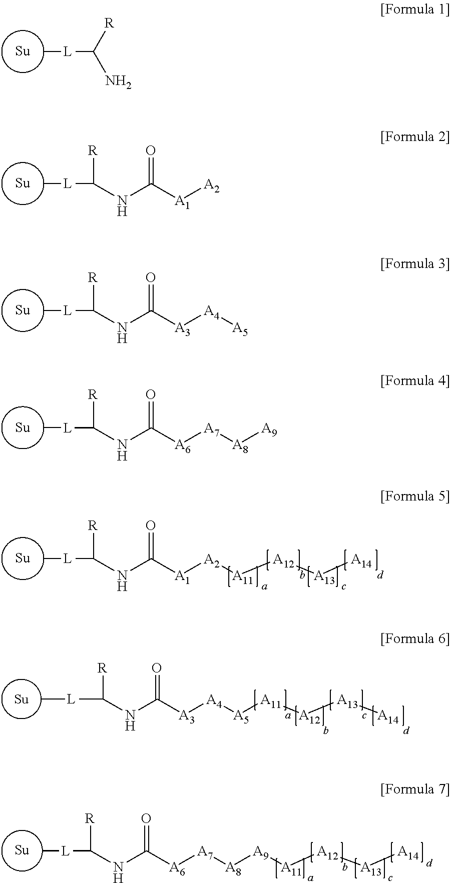

in Formulas 1 to 7,

Su represents a support,

L and R each independently represent hydrocarbyl or heterohydrocarbyl,

A₁ to A₉ and A₁₁ to A₁₄ each independently represent a PNA monomer containing identical or different nucleic acid bases, and

a and b each independently represent an integer of 1, and c and d independently represent an integer of 0 or 1.

As illustrated in FIG. 2, by using a PNA dimer, a PNA trimer, or a PNA tetramer, the method for preparing a PNA oligomer according to an embodiment of the present invention includes a shorter preparation step than a method using a PNA monomer according to the related art, such that the preparation method is very efficient, and separation and purification are also very easily performed. Therefore, a purity of the prepared PNA oligomer is very high.

Specifically, in the method for preparing a PNA oligomer according to an embodiment of the present invention, a PNA dimer, a PNA trimer, or a PNA tetramer that can be synthesized by a solution process is used, such that a PNA oligomer containing n-1 nucleic acid bases or a PNA oligomer containing n-2 nucleic acid bases are not produced as by-products when preparing a desired PNA oligomer containing n nucleic acid bases. Therefore, the PNA oligomer is very easily separated from impurities such as other by-products, and thus, the purity of the PNA oligomer is very high.

According to an embodiment of the present invention, in Formulas 1 to 7, L may be substituted or unsubstituted arylene, alkylene, heteroarylene, or heterocycloalkylene, may be C6-C12 arylene, C1-C10 alkylene, C3-C12 heteroarylene, or C3-C12 heterocycloalkylene, as a specific example, preferably C6-12 arylene, and may be phenylene, as a preferred specific example, and R may be substituted or unsubstituted aryl, alkyl, heteroaryl, or heterocycloalkylene, may be preferably C6-C12 aryl, C1-C10 alkyl, C3-C12 heteroaryl, or C3-C12 heterocycloalkyl, more preferably C1-C10 alkyl C6-C12 aryl, and may be 4-methylphenyl, 2,4-dimethylphenyl, or the like, as an example.

The method for preparing a PNA oligomer of the present invention preferably includes a step A) of obtaining a product that binds a first PNA dimer in which an amine group is protected, a first PNA trimer in which an amine group is protected, or a first PNA tetramer in which an amine group is protected to the structure represented by Formula 1;

a step B) of preparing a deprotected amine product by deprotecting the amine group of the product obtained in the step A); and

a step C) of binding a second PNA dimer in which an amine group is protected, a second PNA trimer in which an amine group is protected, or a second PNA tetramer in which an amine group is protected to the deprotected amine product.

Accordingly, in the method for preparing a PNA oligomer according to an embodiment of the present invention, the PNA dimer, the PNA trimer, or the PNA tetramer is used, such that a PNA oligomer having a significantly high purity due to few by-products may be prepared without performing a capping step of acetylating amine which is a solid-phase unreacted functional group using acetic anhydride or the like.

Preferably, the method for preparing PNA according to an embodiment of the present invention may further include a step of repeatedly performing the step B) and the step C).

In the method for preparing a PNA oligomer according to an embodiment of the present invention, the PNA dimer, the PNA trimer, or the PNA tetramer is used, such that a capping step for protecting an unreacted functional group is not required, unlike the method according to the related art. Therefore, the preparation step may be remarkably reduced, resulting in mass production of the PNA oligomer.

According to an embodiment of the present invention, the PNA oligomer containing a desired number of nucleic acid bases may be easily prepared, and the PNA oligomer may preferably contain four or more nucleic acid bases and may more preferably contain 4 to 40 nucleic acid bases.

According to an embodiment of the present invention, the first PNA dimer, the first PNA trimer, or the first PNA tetramer may be used in an amount of 2 to 7 equivalents with respect to 1 equivalent of an amine functional group of Formula 1, and preferably in an amount of 3 to 5 equivalents with respect to 1 equivalent of a cleavable functional group.

In the method for preparing a PNA oligomer according to an embodiment of the present invention, a small equivalent of the PNA dimer, the PNA trimer, or the PNA tetramer is used, unlike a method for preparing a PNA oligomer using a PNA monomer according to the related art, such that the preparation method according to an embodiment of the present invention is significantly cost-effective and advantageous for commercial applications.

Specifically, in the method for preparing a PNA oligomer using a PNA monomer according to the related art, the PNA oligomer may be prepared by using a PNA monomer in an amount of at least 10 equivalents with respect to 1 equivalent of an amine functional group linked to a support, which is very ineffective and non-cost-effective. On the other hand, in the method for preparing a PNA oligomer according to an embodiment of the present invention, the PNA dimer, the PNA trimer, or the PNA tetramer is used, such that the preparation process is shortened, the PNA dimer, the PNA trimer, or the PNA tetramer is used in a small amount, and the yield of the prepared PNA oligomer is high, which is significantly cost-effective.

According to an embodiment of the present invention, the binding in the step b) may be performed by a coupling reaction.

According to an embodiment of the present invention, the binding in the step b) may be preferably performed by a coupling reaction using N,N,N′,N′-tetramethyl-O-(1H-benzotriazol-1-yl)uronium hexafluorophosphate (HBTU) and benzotriazol-1-yloxy)tripyrrolidinophosphonium hexafluorophosphate (PyBop). According to an embodiment of the present invention, in the binding in the step b), the HBTU and the PyBop are mixed with each other to use as a coupling reagent, such that reactivity between two compounds to be coupled is appropriately adjusted. Therefore, optimal reactivity is maintained in the coupling reaction.

In this regard, according to an embodiment of the present invention, the HBTU and the PyBop may be preferably used in an equivalent ratio of 1:1 to 3.

According to an embodiment of the present invention, the binding in the step b) is preferably performed by the coupling reaction. A solvent used at this time may be a mixed solvent of chlorinated (C1-C4) alkane, dimethylformamide (DMF), and N,N-diisopropylethylamine (DIEA), and the chlorinated (C1-C4) alkane may be one or two or more selected from trichloromethane, dichloromethane, chloromethane, 1,1,2-trichloroethane, 1,1,1-trichloroethane, 1,2-dichloroethane, 1,1-dichloroethane, and chloroethane.

According to an embodiment of the present invention, the mixed solvent contains DMF, DIEA, and chlorinated alkane, such that solubility of a reactant may be appropriately adjusted during the coupling reaction, thereby obtaining a product with a high yield and purity.

Furthermore, according to an embodiment of the present invention, in the mixed solvent, the chlorinated (C1-C4) alkane is used in addition to the DMF, such that a swelling effect of a resin used as a support is significantly improved, thereby increasing the reactivity.

Specifically, according to an embodiment of the present invention, the mixed solvent particularly contains (C1-C4) alkane in addition to the DMF and the DIEA, such that a steric effect of the resin used as a support may be improved and reactivity may be increased due to improved solubility, thereby preparing a product with a high purity.

According to an embodiment of the present invention, the DIEA may be contained in the mixed solvent in an amount of 1 to 10 wt % and preferably 3 to 7 wt % with respect to a total weight of the solvent. A volume ratio of the chlorinated (C1-C4) alkane to the DMF in the mixed solvent may be 1:1 to 2.

Preferably, according to an embodiment of the present invention, the first PNA dimer may contain a nucleic acid base identical to or different from that of the second PNA dimer, the first PNA trimer may contain a nucleic acid base identical to or different from that of the second PNA trimer, and the first PNA tetramer may contain a nucleic acid base identical to or different from that of the second tetramer.

In the method for preparing a PNA oligomer according to an embodiment of the present invention, the PNA oligomer may be prepared by binding the first PNA dimer to the structure represented by Formula 1, specifically, to the amine functional group serving as a cleavable functional group linked to the support and then binding the second PNA dimer, the second PNA trimer, or the second PNA tetramer to the first PNA dimer linked to the amine functional group, the PNA oligomer may be prepared by binding the first PNA trimer to the amine functional group linked to the structure represented by Formula 1 and then binding the second PNA dimer, the second PNA trimer, or the second PNA tetramer to the first PNA trimer linked to the amine functional group, or the PNA oligomer may be prepared by binding the first PNA tetramer to the amine functional group serving as the cleavable functional group linked to the support and then binding the second PNA dimer, the second PNA trimer, or the second PNA tetramer to the first PNA tetramer linked to the amine functional group.

According to an embodiment of the present invention, the nucleic acid base may be adenine, cytosine, 5-methylcytosine, guanine, thymine, uracil, purine, 2,6-diaminopurine, N⁴N⁴-ethanocytosine, N⁶N⁶-ethano-2,6-diaminopurine, 5-(C3-C6)-alkynyluracil, 5-(C3-C6)-alkynyl-cytosine, 5-(1-propargylamino)uracil, 5-(1-propargylamino)cytosine, phenoxazine, 9-aminoethoxyphenoxazine, 5-fluorouracil, pseudoisocytosine, 5-(hydroxymethyl)uracil, 5-aminouracil, pseudouracil, dihydrouracil, 5-(C1-C6)-alkyluracil, 5-(C1-C6)-alkyl-cytosine, 5-(C2-C6)-alkenylcytosine, 5-fluorocytosine, 5-chlorouracil, 5-chlorocytosine, 5-bromouracil, 5-bromocytosine, 7-deazaadenine, 7-deazaguanine, 8-azapurine, 7-deaza-7-substituted purine, thiouracil, or an artificial nucleic acid base, but is not limited thereto.

Preferably, according to an embodiment of the present invention, the nucleic acid base may have one or more amine protective groups. A preferred amine protective group may be fluorenylmethoxycarbonyl (Fmoc), tert-butyloxycarbonyl (Boc), benzyloxycarbonyl (Cbz), benzhydryloxycarbonyl (Bhoc), acetyl, benzoyl, benzyl, carbamate, p-methoxybenzyl, 3,4-dimethoxybenzyl, p-methoxyphenyl, tosyl, trichloroethyl chloroformate, sulfonamides (Nosyl & Nps), or isobutyryl.

Preferably, according to an embodiment of the present invention, the first PNA dimer or the second PNA dimer may be represented by the following Formula 11, the first PNA trimer or the second PNA trimer may be represented by the following Formula 12, and the first PNA tetramer or the second PNA tetramer may be represented by the following Formula 13,

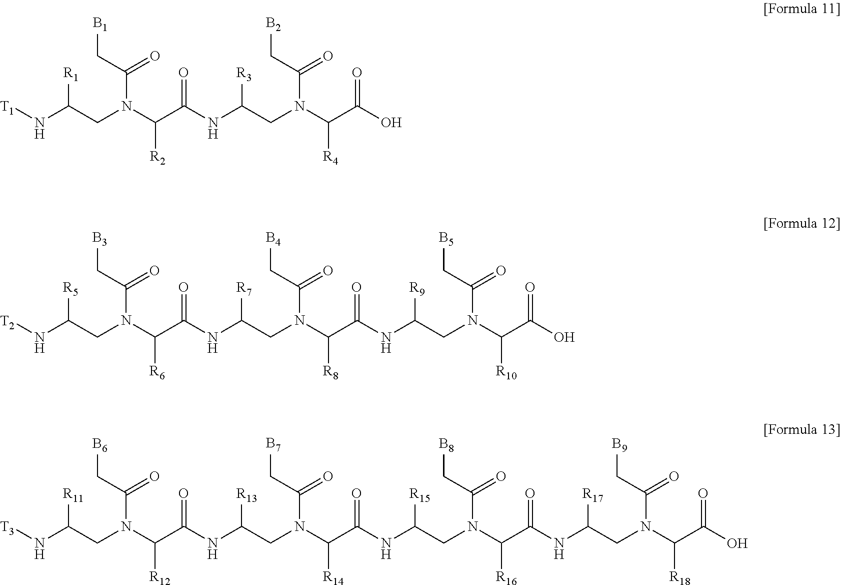

in Formulas 11 to 13,

R₁ to R₁₈ each independently represent hydrogen, an amino acid residue, or an amino acid residue having a substituent,

T₁ to T₃ each independently represent an amine protective group, and

B₁ to B₉ each independently represent a nucleic acid base having or not having an amine protective group.

More specifically, the method for preparing a PNA oligomer according to an embodiment of the present invention may include:

a step A1) of preparing the following Formula 14, 15, or 16 by binding the first PNA dimer represented by Formula 11, the first PNA trimer represented by Formula 12, or the first PNA tetramer represented by Formula 13 to the structure represented by Formula 1;

a step B1) of preparing a deprotected amine product by deprotecting an amine group of Formula 14, 15, or 16; and

a step C1) of preparing a PNA oligomer by reacting the first PNA dimer represented by Formula 11, the first PNA trimer represented by Formula 12, or the first PNA tetramer represented by Formula 13 with the deprotected amine product,

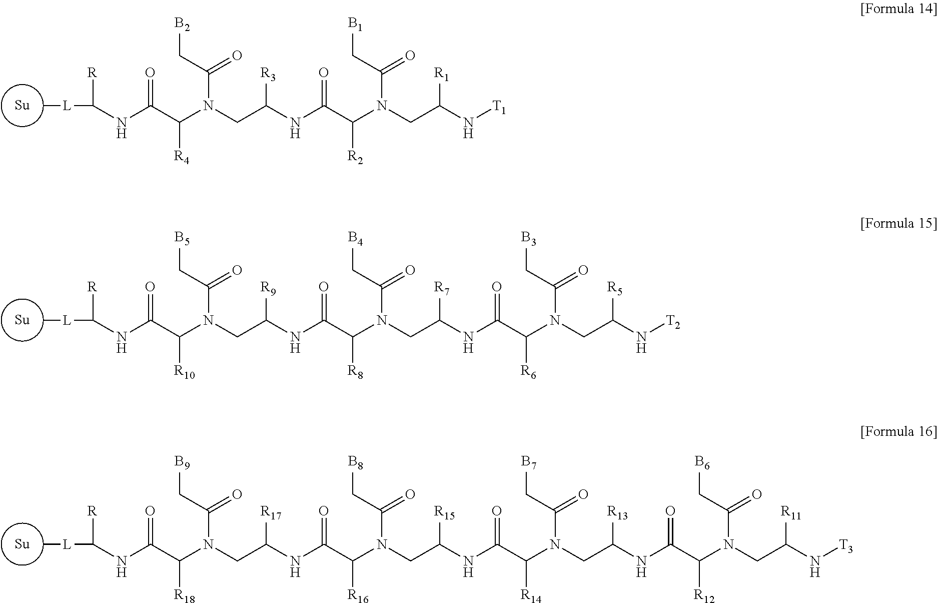

in Formulas 14 to 16,

L and R each independently represent hydrocarbyl or heterohydrocarbyl,

R₁ to R₁₈ each independently represent hydrogen, an amino acid residue, or an amino acid residue having a substituent,

T₁ to T₃ each independently represent an amine protective group, and

B₁ to B₉ each independently represent a nucleic acid base having or not having an amine protective group.

Preferably, according to an embodiment of the present invention, the first PNA dimer, the second PNA dimer, the first PNA trimer, the second PNA trimer, the first PNA tetramer, and the second PNA tetramer may be prepared under a solution process or from a solid phase.

In the method for preparing a PNA oligomer according to the related art, a PNA monomer is used, such that separation is not easily performed when preparing the PNA oligomer. Therefore, the purity of the PNA oligomer is low and the preparation process is lengthened. On the other hand, in the method for preparing a PNA oligomer of the present invention, the PNA dimer, the PNA trimer, or the PNA tetramer is used, such that a PNA oligomer having a high purity may be prepared through a simpler process. However, when a PNA oligomer is prepared using a PNA pentamer, it is disadvantageous in terms of solubility, and thus, it is difficult to prepare the PNA oligomer.

According to an embodiment of the present invention, any material that may be used in the present technical field may be used as the support. Examples thereof may include methylbenzhydrylamine (MBHA), a resin obtained by chloromethylating polystyrene (merrifield resin), a merrifield resin modified with 4-hydroxybenzyl alcohol (wang resin), a Boc-amino acid-linker bonded aminomethyl resin (PAM resin), an N-Fmoc-N-methoxy-linker bonded aminomethyl resin (weinreb resin), a resin obtained by binding p-nitrobenzophenone oxime to polystyrene (oxime resin), and a resin tritylated using polystyrene (trityl resin), and the material may be preferably methylbenzhydrylamine or a trityl resin.

According to an embodiment of the present invention, as a result of high-performance liquid chromatography (HPLC) analysis under the following conditions, the purity of the PNA oligomer may be 60% or higher, preferably 65% or higher, and more preferably 70% or higher.

(Developing solvent: 0.1% TFA in Water, 0.1% TFA in MeCN, gradient condition

UV detector 260 nm, column 250 mm*4.6 mm)

In addition, in the method for preparing a PNA oligomer of the present invention, the PNA oligomer is synthesized without a capping process, and as a result of the high-performance liquid chromatography (HPLC) analysis under the above conditions, the prepared PNA oligomer having the purity of 60% or higher, preferably 65% or higher, and more preferably 70% or higher is provided.

Hereinafter, the present invention will be described in detail with reference to Examples, and the scope of the present invention is not limited by the following Examples.

Material organic solvents used in a reaction were purchased from Novabiochem, Alfa aesar, SAMCHUN CHEMICALS, Junsei chemicals co., Ltd, DUKSAN reagents chemical, and the like, and were used without additional purification. ¹H-NMR analysis of a synthesized compound was performed at room temperature using Bruker 400 or 500 MHz, a solvent in which a ratio of MeCN containing 0.1% TFA to water containing 0.1% TFA was 5:95 was used as an HPLC (waters 1525 Binary hplc pump) developing solvent, the ratio of the developing solvent was gradually changed so that the ratio of MeCN containing 0.1% TFA to water containing 0.1% TFA was changed to 20:80 for 20 minutes, and then a solvent in which a ratio of MeCN containing 0.1% TFA to water containing 0.1% TFA was 95:5 was used for 10 minutes, thereby performing analysis by a column heater 60° C. method.

Nucleic acid bases of the following Formulas 1 to 4 or a nucleic acid base having an amine protective group were prepared in the same manner as that of Korean Patent No. 10-0464261.

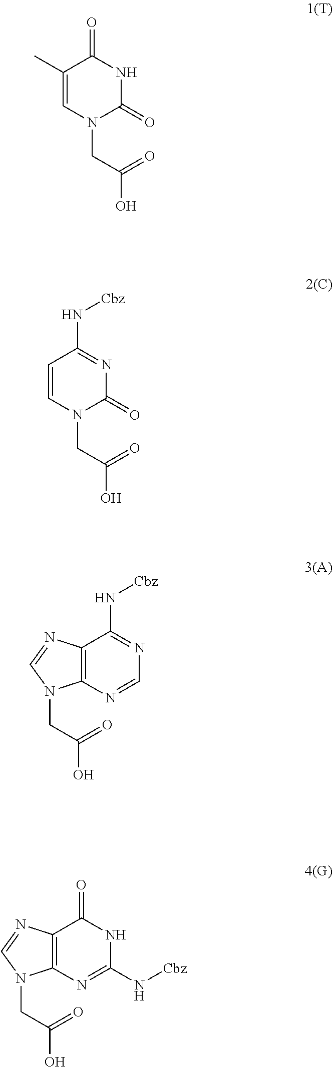 [SMILES: Cc1cn(CC(=O)O)c(=O)[nH]c1=O ; O=C(O)Cn1ccc(NC(=O)OCc2ccccc2)nc1=O ; O=C(O)Cn1cnc2c(NC(=O)OCc3ccccc3)ncnc21 ; O=C(O)Cn1cnc2c(=O)[nH]c(NC(=O)OCc3ccccc3)nc21]

### [Preparation Example 1] Preparation of Compound 5 (Boc-aeg-OEt)

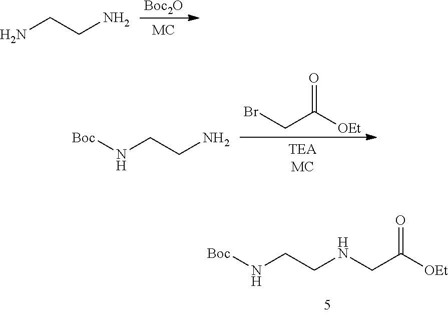

11.0 g (183 mmol) of ethylenediamine was added to a 500 mL Erlenmeyer flask and dissolved in MC. 5.0 g (22.9 mmol) of Boc₂O was dissolved in MC and added dropwise, and stirring was performed at room temperature for 12 hours. After confirming the completion of the reaction using TLC, water was added to extract only an MC layer, and the MC layer was washed with sat. NaCl. After performing a treatment with Na₂SO₄, the solution was filtered and concentrated. MC was added to the concentrated solution and 6.4 mL (45.8 mmol) of triethylamine was added. 2.4 mL (22.0 mmol) of ethyl bromoacetate was dissolved in MC and slowly added dropwise, and the stirring was performed at room temperature for 12 hours. After confirming the completion of the reaction using TLC, water was added to extract only the MC layer. Water was removed with Na₂SO₄ to concentrate the solution, and purification was performed with silica-column chromatography (eluent: EA:Hex=1:1 v/v), thereby obtaining Compound 5 as a clear oil type product (3.79 g, 70%).

### [Preparation Example 2] Preparation of Compound 8 (Boc-Lys(Z)-OMe)

Preparation of Compound 6

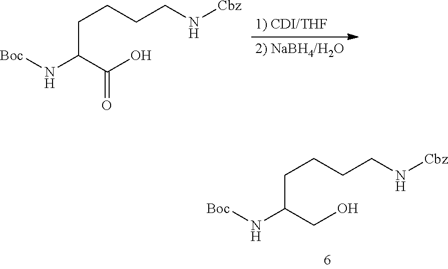 [SMILES: CC(C)(C)OC(=O)NC(CCCCNC(=O)OCc1ccccc1)C(=O)O ; CC(C)(C)OC(=O)NC(CO)CCCCNC(=O)OCc1ccccc1]

5.40 g (14.2 mmol) of Boc-Lys(Z)-COOH was added to a 250 mL 2-neck round-bottom flask under nitrogen and dissolved in 100 mL dry THF. 9.21 g (56.8 mmol) of 1,1′-carbonyldiimidazole was added at once, and stirring was performed at room temperature for 10 minutes. When no more air bubbles were generated, 2.68 g (71.0 mmol) of NaBH₄ was dissolved in 30 mL of distilled water at 0° C. and slowly added, and stirring was performed for 30 minutes. After confirming the completion of the reaction using TLC, the solvent was concentrated. 200 mL of EA was added, the solution was transferred to a separatory funnel and washed with 1 M HCl and then washed with saturated salt water, and then water was removed with sodium sulfate to concentrate the solution. The purification was performed with silica-column chromatography (eluent: EA:HEX=1:1 v/v), thereby obtaining Compound 6 as a clear yellow oil-type product (5.0 g, 96.2%).

Preparation of Compound 7

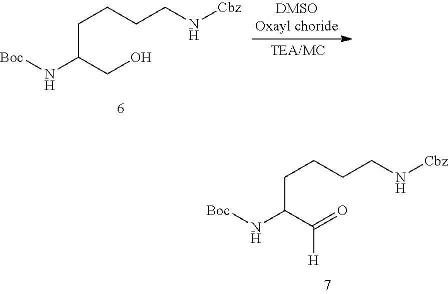 [SMILES: CC(C)(C)OC(=O)NC(CO)CCCCNC(=O)OCc1ccccc1 ; CC(C)(C)OC(=O)NC(C=O)CCCCNC(=O)OCc1ccccc1]

8.71 ml (17.4 mmol) of oxayl chloride was added to a 250 mL 2-neck round-bottom flask under nitrogen using a syringe, and 20 mL of dry MC was added thereto. The temperature was lowered to −20° C. for 5 minutes using NaCl, ice, and methanol, and a solution obtained by mixing 2.47 mL (34.8 mmol) of dry DMSO and 5 mL of dry MC with each other was added dropwise to a 100 mL round-bottom flask. After 5 minutes, 5.80 g (15.8 mmol) of Boc-Lys(Cbz)-OH was dissolved in 20 mL of dry MC and then added to a reaction liquid. After 15 minutes, 15.2 mL (87.1 mmol) of DIEA was added, stirring was performed for 5 minutes, and an ice bath was removed to confirm TLC (EA only, p-Anisaldehyde). After the completion of the reaction, the solution was washed with NaHCO₃, and then the solvent was concentrated, thereby obtaining Compound 7 as a clear oil (5.5 g, 95.3%).

Preparation of Compound 8

Compound 8 was prepared by the same method as in FILBERT TOTSINGAN et. Al. CHIRALITY, 2009, 21, 245-253, and it was confirmed that Compound 8 was synthesized therefrom.

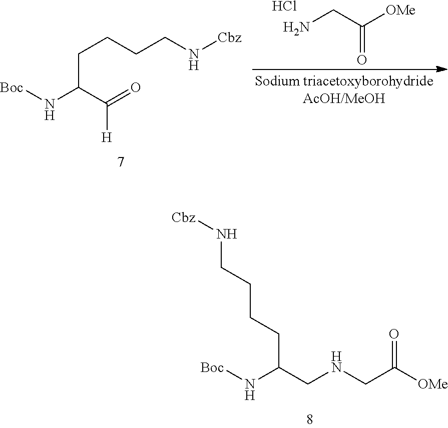 [SMILES: Cl ; CC(C)(C)OC(=O)NC(C=O)CCCCNC(=O)OCc1ccccc1 ; COC(=O)CN ; COC(=O)CNCC(CCCCNC(=O)OCc1ccccc1)NC(=O)OC(C)(C)C]

To a 250 mL round-bottom flask, 5.50 g (15.1 mmol) of Boc-Lys(Cbz)-CHO was added, 50.0 mL of methanol was added, and 2.84 g (22.6 mmol) of Gly-OMe was added. 1.30 mL (22.6 mmol) of acetic acid and 3.94 mL (22.6 mmol) of N,N-diisopropylethylamine were added at 0° C., 9.60 g (45.3 mmol) of sodium triacetoxyborohydride was added, stirring was performed at 0° C. for 2 hours, and then stirring was performed at room temperature overnight. After confirming the reaction of the starting material through the confirmation of TLC (developing solution: EA only, p-anisaldehyde), the solution was concentrated when the reaction was completed, EA was added, the solution was washed with NaHCO₃ and then concentrated, and purification was performed with silica-column chromatography (eluent: EA:HEX=1:1 v/v), thereby obtaining a clear oil-type product (4.5 g, 68.2%).

### [Example 1] Preparation of PNA Monomer

Preparation of Compound 12-3 (Boc-aeg-A(Z)-OEt)

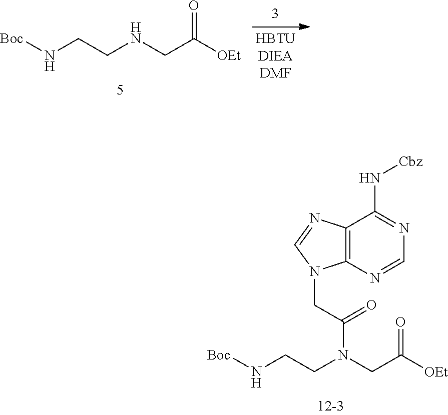 [SMILES: CCOC(=O)CNCCNC(=O)OC(C)(C)C ; CCOC(=O)CN(CCNC(=O)OC(C)(C)C)C(=O)Cn1cnc2c(NC(=O)OCc3ccccc3)ncnc21]

4.67 g (14.3 mmol) of Compound 3 was dissolved in 100 mL of dry N,N-dimethylformamide (DMF). 7.46 mL (42.8 mmol) of N,N-diisopropylethylamine (DIEA) was added, 6.49 g (17.1 mmol) of [0-(1H-benzotriazol-1-yl-N,N,N′,N′-tetramethyluroniumhexafluorophosphate)] (HBTU) was added, and then 3.51 g (14.2 mmol) of Compound 5 was added. After performing stirring for about 1 hour, the completion of the reaction was confirmed using TLC, and DMF was removed. Ethyl acetate (800 mL) was added to a residue from which DMF was removed to dissolve the residue, and an organic layer was washed with saturated NaHCO₃ and saturated salt water using a separatory funnel. Sodium sulfate was added to the organic layer, water in the organic layer was dried for 10 minutes, and then the sodium sulfate was filtered. The filtered solvent was removed using a rotary evaporator. The purification was performed with silica-column chromatography (eluent: 5% MeOH/MC), thereby obtaining Compound 12-3 (4.0 g, 50.4%).

Preparation of Compound 13-3 (Boc-aeg-A(Z)-OH)

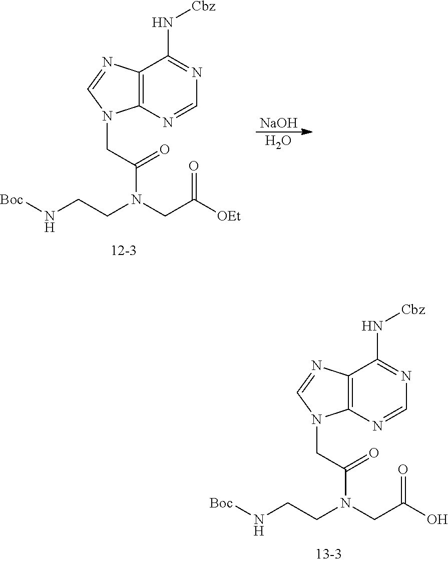

4.80 g (8.64 mmol) of Compound 12-3 was dissolved in 45 mL of 1N-NaOH, stirring was performed for 30 minutes, and then the reaction was completed by performing TLC (developing solution: 5% MeOH/MC) monitoring. The pH was decreased to 2 to 3 with 1 M KHSO₄ at 0° C., the produced solid was filtered with a glass filter and washed with water several times, and the washed solid was dried in P₂O₅, thereby obtaining Compound 13-3 as a while solid-type product (4.30 g, 94.3%).

Preparation of Compound 14-3 (NH2-aeg-A(Z)-OEt)

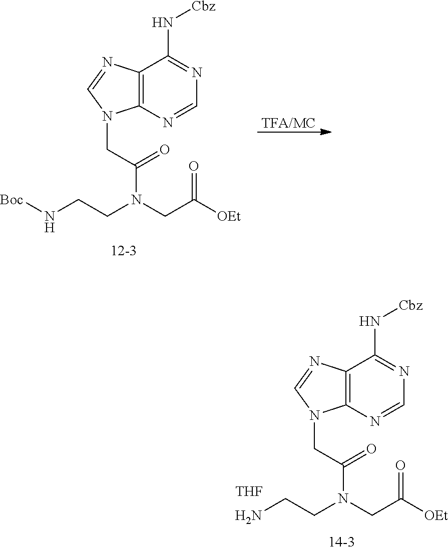 [SMILES: CCOC(=O)CN(CCNC(=O)OC(C)(C)C)C(=O)Cn1cnc2c(NC(=O)OCc3ccccc3)ncnc21 ; "TFA/MC" ; CCOC(=O)CN(CCN)C(=O)Cn1cnc2c(NC(=O)OCc3ccccc3)ncnc21 ; "THF"]

6.30 g (9.5 mmol) of Compound 12-3 was dissolved in 50 mL of 50% TFA/MC, and the reaction was completed by performing TLC monitoring. After the completion of the reaction, a solution was added dropwise to excessive diethyl ether at 0° C. to obtain a precipitate. Filtering was performed, and then Compound 14-3 as a white solid-type product was obtained (4.0 g, 90%).

### [Example 2] Preparation of PNA Monomer

A PNA monomer 13-1 (Boc-aeg-T-OH) and a PNA monomer 14-1 (NH₂-aeg-T-OEt) were prepared in the same manner as that of Example 1, except that Compound 1 was used instead of Compound 3 in the preparation of Compound 12-3 in Example 1.

### [Example 3] Preparation of PNA Monomer

A PNA monomer 13-2 (Boc-aeg-C(Z)-OH), a PNA monomer 14-2 (Boc-aeg-C(Z)-OH), a PNA monomer 13-4 (Boc-aeg-G(Z)-OH), and a PNA monomer 14-4 (NH₂-aeg-G(Z)-OEt) were prepared in the same manner as that of Example 1, except that Compound 2 or 4 was used instead of Compound 3 in the preparation of Compound 12-3 in Example 1.

### [Example 4] Preparation of PNA Monomer

A PNA monomer 13-5 (Boc-Lys(Z)-T-OH) and a PNA monomer 14-5 (NH₂-Lys(Z)-T-OMe) were prepared in the same manner as that of Example 1, except that Compound 8 was used instead of Compound 5 and Compound 1 was used instead of Compound 3 in the preparation of Compound 12-3 in Example 1.

### [Example 5] Preparation of PNA Monomer

Each of PNA monomer 13-6 (Boc-Lys(Z)-C(Z)-OH), a PNA monomer 13-7 (Boc-Lys(Z)-A(Z)-OH), a PNA monomer 13-8 (Boc-Lys(Z)-G(Z)-OH), a PNA monomer 14-6 (NH₂-Lys(Z)-C(Z)-OMe), a PNA monomer 14-7 (NH₂-Lys(Z)-A(Z)-OMe), and a PNA monomer 14-8 (NH₂-Lys(Z)-G(Z)-OMe) was prepared in the same manner as that of Example 4, except that each of Compound 2, 3, or 4 was used instead of Compound 1 in Example 4.

### [Example 6] Preparation of PNA Dimer 16-1 (Boc-AA-OH)

Preparation of Compound 15-1 (Boc-A(Z)A(Z)-OEt)

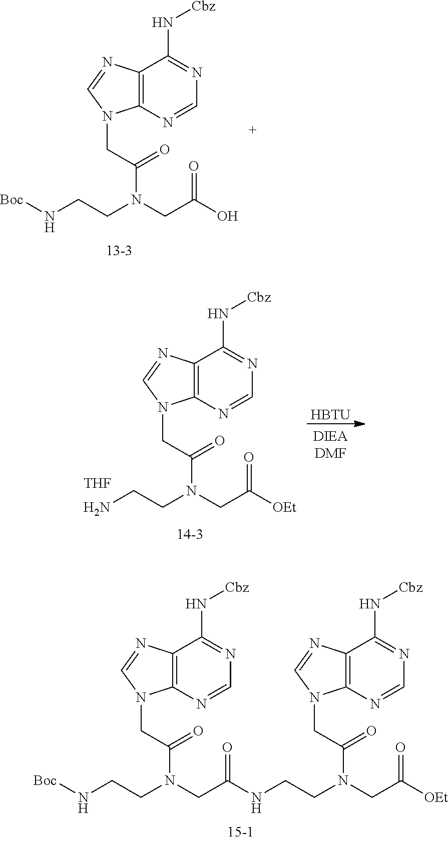

1 g (1.9 mmol) of Compound 13-3 was dissolved in 15 mL of DMF, and then 0.791 g (0.0021 mol) of HBTU and 1.65 mL (0.0095 mol) of DIEA were added. Stirring was performed at room temperature for 10 minutes, 1.05 g (1.9 mmol) of Compound 14-3 was added, and stirring was performed for 3 hours. After confirming the completion of the reaction by performing TLC monitoring, the solvent was completely removed. DCM was added to a residue from which DMF was removed to dissolve the residue, and an organic layer was washed two times with a saturated NaHCO₃ solution (500 mL) using a separatory funnel. Sodium sulfate was added to the organic layer, water in the organic layer was dried for 10 minutes, and then the sodium sulfate was filtered. The filtered solvent was removed, and then purification was performed with silica-column chromatography, thereby obtaining Compound 15-1 (yield: 1.71 g, 93.2%).

Preparation of Compound 16-1 (PNA Dimer Boc-AA-OH)

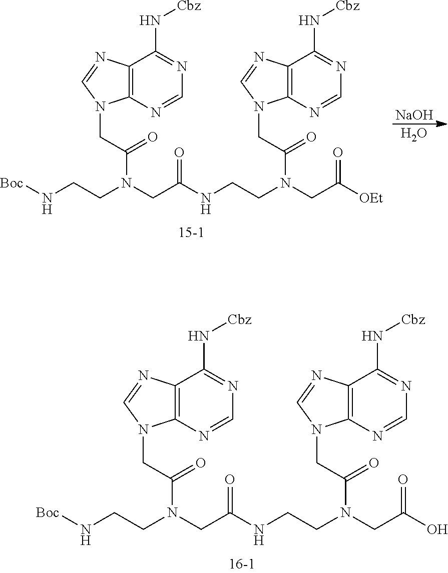

1.71 g (1.9 mmol) of Compound 15-1 was dissolved in 45 mL of 1N-NaOH, stirring was performed for 30 minutes, and then the reaction was completed by performing TLC (developing solution: 10% MeOH/MC) monitoring. The pH was decreased to 2 to 3 with 1 M KHSO₄ at 0° C., the produced solid was filtered with a glass filter and washed with water several times, and the washed solid was dried in P2O5, thereby obtaining White Compound 16-1 (1.59 g, 94.3%).

### [Example 7] Preparation of PNA Dimer

Each of a PNA dimer 16-2 (Boc-TA-OH), a PNA dimer 16-3 (Boc-AT-OH), a PNA dimer 16-4 (Boc-TC-OH), a PNA dimer 16-5 (Boc-GG-OH), a PNA dimer 16-6 (Boc-G″T-OH), and PNA dimers 16-7 (Boc-AC-OH), 16-8 (Boc-CA-OH), 16-9 (Boc-TT-OH), 16-10 (Boc-GT″-OH), 16-11 (Boc-CC″-OH), 16-12 (Boc-T″G-OH), 16-13 (Boc-TA″-OH), and 16-14 (Boc-C″A-OH) was prepared in the same manner as that of Example 6, except that each PNA monomer was changed instead of Compound 13-3 and Compound 14-3 in Example 6. Here, Base″ is NH₂-γLys(Z)-base-OMe.

### [Example 8] Preparation of PNA Trimer

Preparation of Compound 17-1

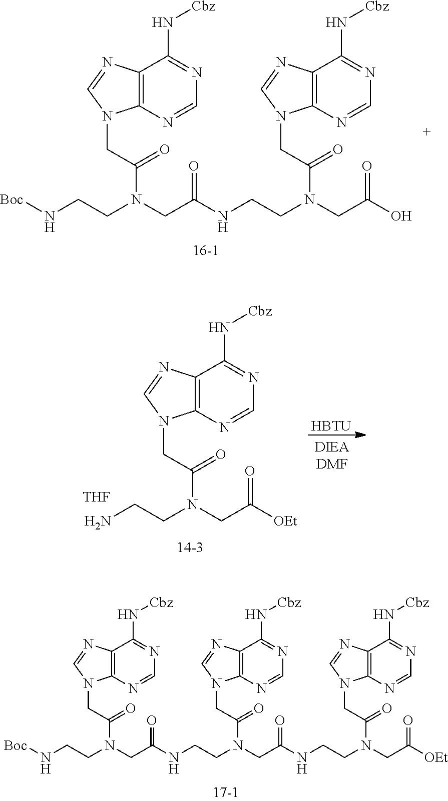

1.56 g (1.7 mmol) of Boc-A(Z)A(Z)-OEt (16-1) was dissolved in 15 mL of DMF, and then 0.695 g (1.8 mmol) of HBTU and 1.45 mL (9.5 mmol) of DIEA were added. Stirring was performed at room temperature for 10 minutes, 0.95 g (1.7 mmol) of NH2-aeg-A(Z)-OEt (14-3) was added, and stirring was performed for 3 hours. After confirming the completion of the reaction by performing TLC monitoring, the solvent was completely removed. DCM was added to a residue from which DMF was removed to dissolve the residue, and an organic layer was washed two times with a saturated NaHCO₃ solution (500 mL) using a separatory funnel. Sodium sulfate was added to the organic layer, water in the organic layer was dried for 10 minutes, and then the sodium sulfate was filtered. The filtered solvent was removed, and then purification was performed with silica-column chromatography, thereby obtaining Boc-aeg-A(Z)A(Z) A(Z)-OEt (17-1) (yield: 2.17 g, 93.1%).

Preparation of Compound 18-1 (PNA Trimer)

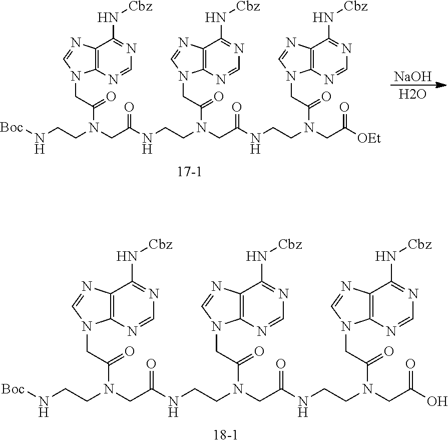

1.5 g (1.1 mmol) of Compound 17-3 was dissolved in 30 mL of 1N-NaOH, stirring was performed for 30 minutes, and then the reaction was completed by performing TLC (developing solution: 10% MeOH/MC) monitoring. The pH was decreased to 2 to 3 with 1 M KHSO₄ at 0° C., the produced solid was filtered with a glass filter and washed with water several times, and the washed solid was dried in P₂O₅, thereby obtaining White Compound 18-1 (1.35 g, 93.9%).

### [Example 9] Preparation of PNA Trimer

A PNA trimer 18-2 (Boc-TAT-OH), a PNA trimer 18-3 (Boc-ATC)-OH), a PNA trimer 18-4 (Boc-TCG-OH), a PNA trimer 18-5 (Boc-GGT-OH), a PNA trimer 18-6 (Boc-TCC-OH), and PNA trimers 18-7 (Boc-G″TG-OH), 18-8 (Boc-ACA-OH), 18-9 (Boc-TTA-OH), 18-10 (Boc-GT″G-OH), and 18-11 (Boc-C″AT-OH) were prepared in the same manner as that of Example 11, except that different PNA dimers and PNA monomers were used instead of Compound 16-1 and Compound 14-3 in Example 8. Here, Base″ is NH₂-γLys(Z)-base-OMe.

### [Example 10] Preparation of PNA Tetramer

Preparation of Compound 19-1

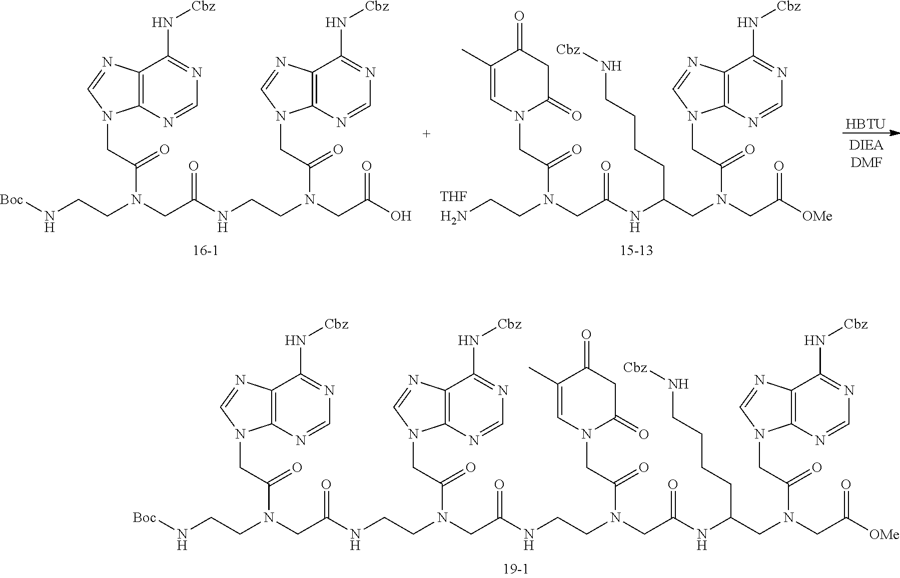

1.56 g (1.7 mmol) of Boc-A(Z)A(Z)-OEt (16-1) was dissolved in 15 mL of DMF, and then 0.943 g (2.5 mmol) of HBTU and 5.78 mL (33.2 mmol) of DIEA were added. Stirring was performed at room temperature for 10 minutes, 1.7 g (1.7 mmol) of THF.NH₂-TA″-OMe (15-13) was added, and stirring was performed for 3 hours. After confirming the completion of the reaction by performing TLC monitoring, the solvent was completely removed. DCM was added to a residue from which DMF was removed to dissolve the residue, and an organic layer was washed two times with a saturated NaHCO₃ solution (500 mL) using a separatory funnel. Sodium sulfate was added to the organic layer, water in the organic layer was dried for 10 minutes, and then the sodium sulfate was filtered. The filtered solvent was removed, and then purification was performed with silica-column chromatography, thereby obtaining Boc-aegA(Z)A(Z)T-γLys(Z)A(Z)-OMe (19-1) (yield: 2.1 g, 68.7%).

Preparation of Compound 20-1

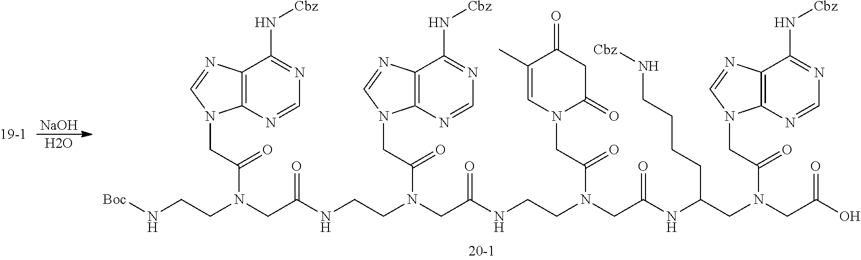

2.1 g (1.1 mmol) of Compound 19-1 was dissolved in 10 mL of 1N-NaOH, stirring was performed for 30 minutes, and then the reaction was completed by performing TLC (developing solution: 20% MeOH/MC) monitoring. The pH was decreased to 2 to 3 with 1 M KHSO₄ at 0° C., the produced solid was filtered with a glass filter and washed with water several times, and the washed solid was dried in P₂O₅, thereby obtaining Compound 19-2 (1.9 g, 91.2%).

### [Example 11] Preparation of PNA Tetramer

A PNA tetramer 20-2 (Boc-G″TAA-OH) and a PNA tetramer 20-3 (Boc-AACC″-OH) were prepared in the same manner as that of Example 10, except that different PNA dimers were used instead of Compound 16-1 and Compound 15-13 in Example 10. Here, Base″ is NH₂-γLys(Z)-base-OMe.

### [Example 12] Preparation of PNA Oligomer (NH2-ATC TCG TAT-H) Using PNA Trimer 1

An MBHA resin (100-200 mesh, Novabiochem) was swollen in 1,2-dichloroethane (DCM) for 30 minutes. An amine group of the swollen MBHA resin was activated (free amine) with a 5% DIEA/DMF solution, and then impurities were removed by performing washing with DMF three times. A PNA trimer (TAT) dissolved in DMF and DCE (volume ratio of 2:1) was added in an amount of 3 equivalents with respect to 1 equivalent of the amine functional group of MBHA, HATU and PyBop (equivalent ratio of 1:1) were added to DIEA so that the amount thereof was 5 wt % with respect to a total weight of the mixed solvent, and then a coupling reaction was performed at room temperature for 3 hours. After the reaction was completed, impurities were removed by performing washing with DMF three times, a 5% TFA/DCM solution was added to deprotect a Boc protective group, and then impurities were removed by performing washing with DMF three times again. In the same manner as described above, each of a step of adding each of trimers TCG and ATC dissolved in DMF and DCE (volume ratio of 2:1), a step of adding HATU and PyBop (equivalent ratio of 1:1) to DIEA, and a step of performing a coupling reaction at room temperature for 3 hours was repeatedly performed, thereby preparing a PNA oligomer containing 9 nucleic acid bases (ATC TCG TAT).

Thereafter, a TFMSA/TFA/m-cresol (2:8:1) solution was added, a deresinated reaction was performed at room temperature for 2 hours, and then the reaction solution was filtered. Thereafter, the resin was washed with TFA, a filtrate and a wash liquid were mixed with each other, and diethyl ether was added thereto to precipitate a deresinated oligomer. A supernatant was removed using centrifugation, and the remaining precipitate was washed with diethyl ether and dried, thereby preparing a PNA oligomer containing 9 nucleic acid bases.

A crude purity was measured with HPLC (waters 1525 Binary hplc pump, 5% MeCN 20% for 20 mins 95% for 30 mins) of the prepared crude PNA oligomer.

The crude purity was 91.98% when synthesizing a 9mer PNA oligomer using trimers, and it could be confirmed that the PNA oligomer with the high purity was synthesized.

### [Comparative Example 1] Preparation of PNA Oligomer (NH2-ATC TCG TAT-H) Using PNA Monomer

A PNA oligomer NH2-ATC TCG TAT-H was prepared using a PNA monomer in the same manner as that of Curr Protoc Nucleic Acid Chem. 2002 August; Chapter 4: Unit 4.11.

As a result of measuring a crude purity with HPLC (waters 1525 Binary hplc pump, 5% MeCN 20% for 20 mins 95% for 30 mins) of the prepared crude PNA oligomer, the crude purity was 63.31%, and it could be seen that the purity was significantly lower than that of the PNA oligomer prepared in Example 12 of the present invention.

### [Example 13] Preparation of PNA Oligomer (NH2-TAT ATC TCG TAT-H) Using PNA Trimer

A desired PNA oligomer was prepared in the same manner as that of Example 12, except that the types of the PNA trimer were changed in Example 12.

A crude purity was measured with HPLC (waters 1525 Binary hplc pump, 5% MeCN 20% for 20 mins 95% for 30 mins) of the prepared crude PNA oligomer.

The crude purity was 80.01% when synthesizing a 12mer PNA oligomer using trimers, and it could be confirmed that the PNA oligomer with the high purity was synthesized.

### [Comparative Example 2] Preparation of PNA Oligomer (NH2-TAT ATC TCG TAT-H) Using PNA Monomer

A desired PNA oligomer was prepared in the same manner as that of Comparative Example 1, except that the types of the PNA monomer were changed in Comparative Example 1.

As a result of measuring a crude purity with HPLC (waters 1525 Binary hplc pump, 5% MeCN 20% for 20 mins 95% for 30 mins) of the prepared crude PNA oligomer, the crude purity was 46.59%, and it could be seen that the purity was significantly lower than that of the PNA oligomer prepared in Example 13 of the present invention.

### [Example 14] Preparation of PNA Oligomer (NH2-GGT-TCC-G″TG-CA-ACA-TC-H) Using PNA Trimer and Dimer

A desired PNA oligomer was prepared in the same manner as that of Example 12, except that the types of the PNA trimer and dimer were changed in Example 12.

HPLC of the prepared PNA oligomer was measured. As a result, it could be seen that the crude purity of the PNA oligomer was 71.5%.

### [Example 15] Preparation of PNA Oligomer (NH2-TCC TTA GT″G GT″G TCC-H) Using PNA Trimer

A desired PNA oligomer was prepared in the same manner as that of Example 12, except that the types of the PNA trimer and dimer were changed in Example 12. However, a 12mer PNA oligomer was synthesized instead of the 9mer PNA oligomer and then a part of the reaction liquid was obtained to perform the deresinated reaction, thereby preparing the 12mer PNA oligomer. PNA trimers were used for some 12mer PNA oligomers to prepare a 15mer PNA oligomer.

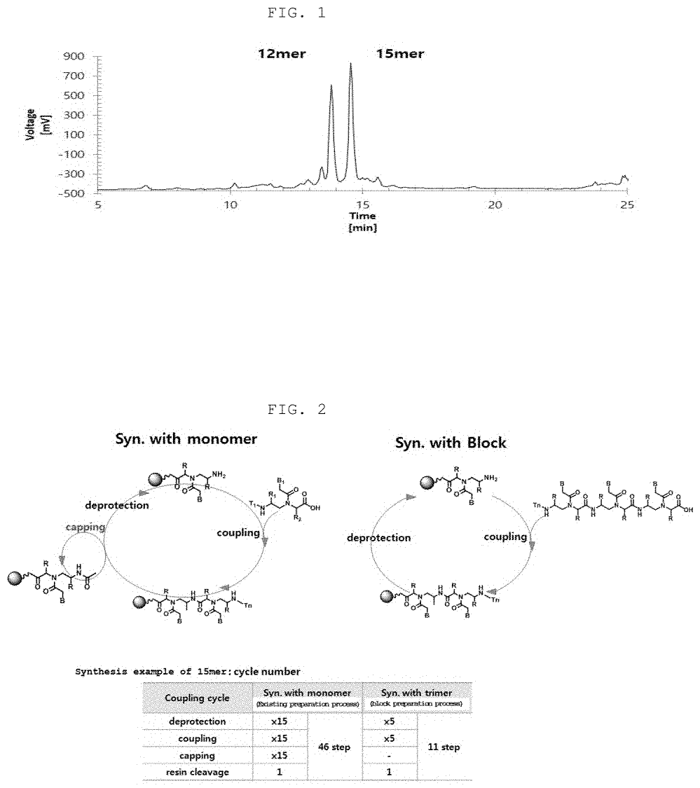

HPLC analysis was performed using a mixed solution of the 12mer PNA oligomer and 15mer PNA oligomer prepared in the above. As illustrated in FIG. 1, it could be seen that the prepared 15mer PNA oligomer was easily purified when performing large-scale synthesis because the 15mer PNA oligomer was easily separated due to a large difference in retention time between the PNA oligomer containing 12 nucleic acid bases which were by-products and the 15mer PNA oligomer.

That is, it could be seen that the 12mer PNA by-products which may be generated when synthesizing the PNA oligomer using the trimers of the present invention were easily purified, but the 14mer and 13mer oligomers which were by-products which may be generated when synthesizing the PNA oligomer using the monomers were not easily purified.

### [Example 16] Preparation of PNA Oligomer (NH2-AATA″ G″TAA-H) Using PNA Tetramer

An MBHA resin (100-200 mesh, Novabiochem) was swollen in DCM for 30 minutes. An amine group of the swollen MBHA resin was activated (free amine) with a 5% DIEA/DMF solution, and then impurities were removed by performing washing with DMF three times. 3 equivalents of a PNA tetramer (G″TAA) dissolved in DMF was added thereto, HATU and DIEA were added, and a coupling reaction was performed at room temperature for 3 hours. After the reaction was completed, impurities were removed by performing washing with DMF three times, a 5% TFA/DCM solution was added to deprotect a Boc protective group, and then impurities were removed by performing washing with DMF three times again. Each of a step of adding a tetramer (AATA″) dissolved in DMF, a step of adding HATU and DIEA, and a step of performing a coupling reaction at room temperature for 3 hours was repeatedly performed, thereby preparing a PNA oligomer containing 8 nucleic acid bases (ATC TCG TAT).

Thereafter, a TFMSA/TFA/m-cresol (2:8:1) solution was added, a deresinated reaction was performed at room temperature for 2 hours, and then the reaction solution was filtered. Thereafter, the resin was washed with TFA, a filtrate and a wash liquid were mixed with each other, and diethyl ether was added thereto to precipitate a deresinated oligomer. A supernatant was removed using centrifugation, and the remaining precipitate was washed with diethyl ether and dried, thereby preparing a PNA oligomer containing 8 nucleic acid bases.

A crude purity was measured with HPLC (waters 1525 Binary hplc pump, 5% MeCN 20% for 20 mins 95% for 30 mins) of the prepared crude PNA oligomer.

The crude purity of the 8mer PNA oligomer obtained by using the tetramers was 39.3%.

### [Example 17] Preparation of PNA Oligomer (NH2-AATA″ G″TAA-H) Using PNA Tetramer

A 8mer PNA oligomer was prepared in the same manner as that of Example 16, except that a mixed solvent obtained by adding N,N-diisopropylethylamine (DIEA) to a solution in which dimethylformamide (DMF) and 1,2-dichloroethylene (DCE) were mixed with each other in a volume ratio of 2:1 so that the amount of DIEA was 5 wt % was used instead of DMF as the solvent in which the PNA tetramer was dissolved in Example 16. The crude purity of the prepared PNA oligomer was 61.8%.

### [Example 18] Preparation of PNA Oligomer (NH2-AACC″/C″AT/T″A-H) Using PNA Tetramer, Trimer, and Dimer of the Present Invention

An MBHA resin (100-200 mesh, Novabiochem) was swollen in DCM for 30 minutes. An amine group of the swollen MBHA resin was activated (free amine) with a 5% DIEA/DMF solution, and then impurities were removed by performing washing with DMF three times. A PNA dimer (T″A) dissolved in DMF and DCE (volume ratio of 2:1) was added in an amount of 3 equivalents with respect to 1 equivalent of the amine functional group of MBHA, and HATU and PyBop (equivalent ratio of 1:1) were added to DIEA so that the amount thereof was 5 wt % with respect to a total weight of the mixed solvent, and then a coupling reaction was performed at room temperature for 3 hours. After the reaction was completed, impurities were removed by performing washing with DMF three times, a 5% TFA/DCM solution was added to deprotect a Boc protective group, and then impurities were removed by performing washing with DMF three times again. In the same manner as described above, each of a step of adding each of trimers (C″AT) and tetramers AACC″ dissolved in DMF and DCE (volume ratio of 2:1), a step of adding HATU and PyBop (equivalent ratio of 1:1) to DIEA, and a step of performing a coupling reaction at room temperature for 3 hours was repeatedly performed, thereby preparing a PNA oligomer containing 9 nucleic acid bases (ATC TCG TAT).

Thereafter, a TFMSA/TFA/m-cresol (2:8:1) solution was added, a deresinated reaction was performed at room temperature for 2 hours, and then the reaction solution was filtered. Thereafter, the resin was washed with TFA, a filtrate and a wash liquid were mixed with each other, and diethyl ether was added thereto to precipitate a deresinated oligomer. A supernatant was removed using centrifugation, and the remaining precipitate was washed with diethyl ether and dried, thereby preparing a PNA oligomer containing 9 nucleic acid bases.

A crude purity was measured with HPLC (waters 1525 Binary hplc pump, 5% MeCN 20% for 20 mins 95% for 30 mins) of the prepared crude PNA oligomer.

The crude purity of the 8mer PNA oligomer obtained by using the tetramers was 33.8%.

### [Example 19] Preparation of PNA Oligomer (NH2-AACC″/C″AT/T″A-H) Using PNA Tetramer, Trimer, and Dimer of the Present Invention

A 9mer PNA oligomer was prepared in the same manner as that of Example 18, except that benzotriazol-1-yloxy)tripyrrolidinophosphonium hexafluorophosphate (PyBop) was used as the coupling reagent in Example 18. The crude purity of the prepared PNA oligomer was 70.9%.

### [Example 20] Preparation of PNA Oligomer (NH2-AATA″ G″TAA-H) Using PNA Tetramer

A 9mer PNA oligomer was prepared in the same manner as that of Example 18, except that 0.3 equivalents of N,N,N′,N′-Tetramethyl-O-(1H-benzotriazol-1-yl)uronium hexafluorophosphate (HBTU) and 0.9 equivalents of (benzotriazol-1-yloxy)tripyrrolidinophosphonium hexafluorophosphate) (PyBop) were used as the coupling reagent in Example 18. The crude purity of the prepared PNA oligomer was 76.3%.

### [Example 21] Preparation of PNA Oligomer (NH2-AATA″ G″TAA-H) Using PNA Tetramer

A 8mer PNA oligomer was prepared in the same manner as that of Example 18, except that 0.5 equivalents of N,N,N′,N′-Tetramethyl-O-(1H-benzotriazol-1-yl)uronium hexafluorophosphate (HBTU) and 0.7 equivalents of (benzotriazol-1-yloxy)tripyrrolidinophosphonium hexafluorophosphate) (PyBop) were used as the coupling reagent in Example 18. The crude purity of the prepared PNA oligomer was 88.3%.

### [Example 22] Preparation of PNA Oligomer (NH2-AATA″ G″TAA-H) Using PNA Tetramer

A 8mer PNA oligomer was prepared in the same manner as that of Example 18, except that (1-ethyl-3-(3-dimethylaminopropyl)carbodiimide hydrochloride) (EDC) was used as the coupling reagent in Example 18. The crude purity of the prepared PNA oligomer was 25%.

As shown in Examples 16 and 17, it could be seen that the purity of the PNA oligomer was higher than the case where the mixed solvent of chlorinated (C1-C4) alkane, DMF, and DIEA was used.

In addition, as shown in Examples 18 to 22, it could be seen that the purity of the PNA oligomer obtained by mixing the coupling reagent was increased, and in particular, the HBTU and the PyBop were used in an equivalent ratio of 1:1 to 3, such that the PNA oligomer having an extremely high purity was prepared.

1. A method for preparing a PNA oligomer, comprising the steps of:
(a) preparing a structure represented by the following Formula 2, 3, or 4 by binding a first PNA dimer, a first PNA trimer, or a first PNA tetramer to a structure represented by the following Formula 1; and
(b) preparing a PNA oligomer represented by the following Formula 5, 6, or 7 by binding a second PNA dimer, a second trimer PNA, and a second tetramer PNA to the structure represented by Formula 2, 3, or 4, 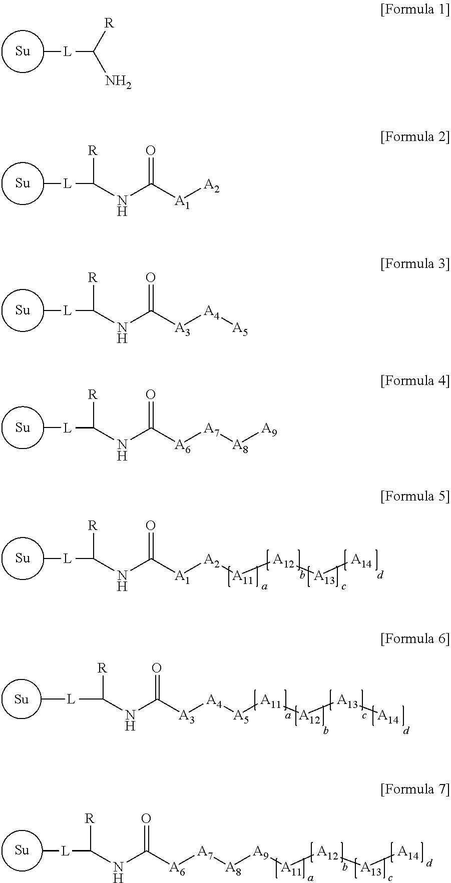
in Formulas 1 to 7,
Su represents a support,
L and R each independently represent hydrocarbyl or heterohydrocarbyl,
A₁ to A₉ and A₁₁ to A₁₄ each independently represent a PNA monomer containing identical or different nucleic acid bases, and
a and b each independently represent an integer of 1, and c and d independently represent an integer of 0 or 1.

2. The method of claim 1, wherein the preparation method includes the steps of:
(A) obtaining a product by binding a first PNA dimer in which an amine group is protected, a first PNA trimer in which an amine group is protected, or a first PNA tetramer in which an amine group is protected to the structure represented by Formula 1;
(B) preparing a deprotected amine product by deprotecting the amine group of the product obtained in step (A); and
(C) binding a second PNA dimer in which an amine group is protected, a second PNA trimer in which an amine group is protected, or a second PNA tetramer in which an amine group is protected to the deprotected amine product.

3. The method of claim 2, further comprising a step of repeatedly performing step (B) and step (C).

4. The method of claim 1, wherein the PNA oligomer contains four or more nucleic acid bases.

5. The method of claim 1, wherein the first PNA dimer, the first PNA trimer, or the first PNA tetramer is used in an amount of 2 to 5 equivalents with respect to 1 equivalent of an amine functional group of Formula 1.

6. The method of claim 1, wherein the binding in 4 the step (b) is performed by a coupling reaction using N,N,N′,N′-tetramethyl-O-(1H-benzotriazol-1-yl)uronium hexafluorophosphate (HBTU) and (benzotriazol-1-yloxy)tripyrrolidinophosphonium (PyBop).

7. The method of claim 6, wherein the HBTU and the PyBop are used in an equivalent ratio of 1:1 to 3.

8. The method of claim 1, wherein the binding in step (b) is performed in a mixed solvent of chlorinated (C1-C4) alkane, dimethylformamide (DMF), and N,N-diisopropylethylamine (DIEA).

9. The method of claim 8, wherein the chlorinated (C1-C4) alkane is one or two or more selected from trichloromethane, dichloromethane, chloromethane, 1,1,2-trichloroethane, 1,1,1-trichloroethane, 1,2-dichloroethane, 1,1-dichloroethane, and chloroethane.

10. The method of claim 8, wherein the DIEA is contained in the mixed solvent in an amount of 1 to 10 wt % with respect to a total weight of the mixed solvent.

11. The method of claim 10, wherein a volume ratio of the chlorinated (C1-C4) alkane to the DMF in the mixed solvent is 1:1 to 2.

12. The method of claim 1, wherein the first PNA dimer contains a nucleic acid base identical to or different from that of the second PNA dimer,
the first PNA trimer contains a nucleic acid base identical to or different from that of the second PNA trimer, and
the first PNA tetramer contains a nucleic acid base identical to or different from that of the second PNA tetramer.

13. The method of claim 12, wherein the nucleic acid base is adenine, cytosine, 5-methylcytosine, guanine, thymine, uracil, purine, 2,6-diaminopurine, N⁴N⁴-ethanocytosine, N⁶N⁶-ethano-2,6-diaminopurine, 5-(C3-C6)-alkynyluracil, 5-(C3-C6)-alkynyl-cytosine, 5-(1-propargylamino)uracil, 5-(1-propargylamino)cytosine, phenoxazine, 9-aminoethoxyphenoxazine, 5-fluorouracil, pseudoisocytosine, 5-(hydroxymethyl)uracil, 5-aminouracil, pseudouracil, dihydrouracil, 5-(C1-C6)-alkyluracil, 5-(C1-C6)-alkyl-cytosine, 5-(C2-C6)-alkenylcytosine, 5-fluorocytosine, 5-chlorouracil, 5-chlorocytosine, 5-bromouracil, 5-bromocytosine, 7-deazaadenine, 7-deazaguanine, 8-azapurine, 7-deaza-7-substituted purine, thiouracil, or an artificial nucleic acid base.

14. The method of claim 12, wherein the nucleic acid base has one or more amine protective groups.

15. The method of claim 13, wherein the protective group is fluorenylmethoxycarbonyl, tert-butyloxycarbonyl, benzyloxycarbonyl, benzhydryloxycarbonyl (Bhoc), acetyl, benzoyl, benzyl, carbamate, p-methoxybenzyl, 3,4-dimethoxybenzyl, p-methoxyphenyl, tosyl, trichloroethyl chloroformate, sulfonamides, or isobutyryl.

16. The method of claim 1, wherein the first PNA dimer or the second PNA dimer is represented by the following Formula 11,
the first PNA trimer or the second PNA trimer is represented by the following Formula 12, and
the first PNA tetramer or the second PNA tetramer is represented by the following Formula 13, 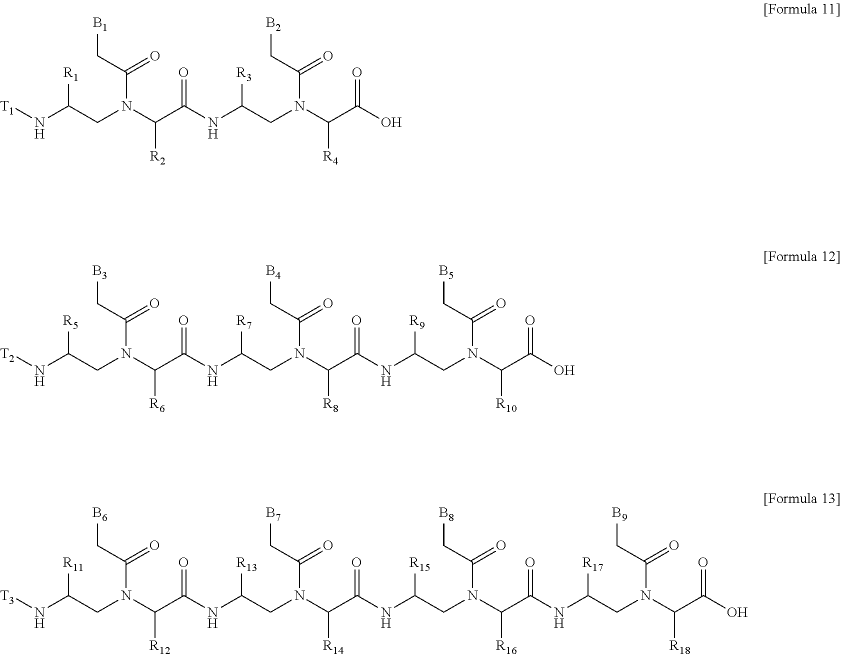
in Formulas 11 to 13,
R₁ to R₁₈ each independently represent hydrogen, an amino acid residue, or an amino acid residue having a substituent,
T₁ to T₃ each independently represent an amine protective group, and
B₁ to B₉ each independently represent a nucleic acid base having or not having an amine protective group.

17. The method of claim 1, wherein the first PNA dimer, the first PNA trimer, the second PNA trimer, the first PNA tetramer, and the second PNA tetramer are prepared under a solution process or from a solid phase.

18. The method of claim 1, wherein the support is methylbenzhydrylamine, a resin obtained by chloromethylating polystyrene, a merrifield resin modified with 4-hydroxybenzyl alcohol, a Boc-amino acid-linker bonded aminomethyl resin, an N-Fmoc-N-methoxy-linker bonded aminomethyl resin, a resin obtained by binding p-nitrobenzophenone oxime to polystyrene, or a resin tritylated using polystyrene.

19. The method of claim 1, wherein when the number of nucleic acid bases of the prepared PNA oligomer is n, impurities of PNA oligomers containing n-1 and n-2 nucleic acid bases are not present.
